(19) United States
(12) Patent Application Publication
(10) Pub. No.: US 2011/0207999 A1
(43) Pub. Date: Aug. 25, 2011

(54) TORQUE TRANSMISSION DEVICE HAVING CONTROL WIRE

(76) Inventors: Nobuyuki Torisawa, Kanagawa (JP); Motohiko Matsushita, Kanagawa (JP); Teruyuki Emura, Kanagawa (JP)

(21) Appl. No.: 12/982,279

(22) Filed: Dec. 30, 2010

(30) Foreign Application Priority Data
Feb. 24, 2010 (JP) 2010-039440

(51) Int. Cl. A61B 1/01; F16C 1/10; F16C 1/12

(52) U.S. Cl. 600/114; 74/502.4; 74/501.6

(57) ABSTRACT

A torque transmission device is a steerable device for an endoscope, and includes a first link element having a first guide lumen. A second link element is disposed at a distal end of the first link element in a longitudinal axis direction, and has a second guide lumen. A control wire is disposed through the first and second guide lumens movably in the longitudinal axis direction. A pivot mechanism couples the first link element to the second link element in a rotatable manner about a pivot axis being perpendicular to the longitudinal axis direction. The control wire through the first and second guide lumens extends in a coplanar manner with the pivot axis and crosswise thereto. Preferably, there is a flexible section having the first and second link elements. A steering unit has third and fourth link elements.

### BACKGROUND OF THE INVENTION

1. Field of the Invention

The present invention relates to a torque transmission device having a control wire. More particularly, the present invention relates to a torque transmission device in which torque can be transmitted with exactitude by removing unwanted influence of tension applied to a control wire.

2. Description Related to the Prior Art

Laparoscopic surgery is known in which a medical instrument or endoscope is entered into a body cavity percutaneously through incisions formed in abdominal skin at a size of several millimeters from tens of millimeters. A doctor or operator observes an image obtained by the endoscope by use of a display panel, and manipulates the medical instrument to conduct the laparoscopic surgery. In U.S. Pat. No. 7,637,905 (corresponding to JP-A 2006-516910), the medical instrument for use in the laparoscopic surgery has a guide tube. The guide tube only can be moved back and forth longitudinally and moved rotationally. It is difficult to handle the medical instrument for highly complicated treatment for affected tissue.

A tool arm has been developed to enable complicated movement of the medical instrument required for treatment of the affected tissue. The tool arm includes an arm and an end effector disposed at an end of the arm, having a proximal end and a distal end, the proximal end being shaped to form a curve laterally, the distal end being operated to bend inwards in the lateral direction, so as to form an angular shape or boomerang shape. See U.S. Pat. No. 7,637,905. The tool arm is operable for any of various directions, and is rotatable for complicated movement to capture the tissue, pull the tissue, apply tension to the tissue, and raise the tissue.

The tool arm is inserted through an overtube or shaft or jacket tube for use, and includes a handle section or proximal end portion, a steering unit or distal end portion, and a flexible section or shaft disposed between the handle section and the steering unit.

The steering unit is bent in one direction by pull of a pull wire extending through a lumen upon manipulating a wire puller or cuff on the handle section. When the handle section is rotated, its torque is transmitted by the flexible section to the steering unit. Thus, the handle section can be adjusted for a direction as desired for steering.

The steering unit includes a plurality of link elements and pivot mechanisms. The link elements are arranged serially. Each of the pivot mechanisms interconnects adjacent two of the link elements in a rotatable manner so as to keep the series of the link elements bendable. See U.S. Pat. No. 7,637,905. Specifically, only one end of each of the link elements is used for coupling of the pivot mechanisms. Remaining portions of the link elements beside the one end has recesses as gaps for the pivot mechanisms to bend with sufficient play. A guide lumen is formed in each of the link elements, shaped circularly as viewed in a cross section, for passage of a tool or instrument.

In U.S. Pat. No. 7,637,905, the flexible section is a series of the link elements connected with one another, and has an easy bendable form. Each of the link elements has a first end and a second end. In a manner similar to the link elements in the steering unit of the endoscope, a projection is formed on the first end. A recess is formed in the second end. The projection and the recess are arcuate, and engaged with one another to for interconnection of the link elements in a rotatable manner about a pivot axis which extends in a direction of projecting the projection or a direction of retraction of the recess. The pivot mechanisms are so arranged that the projection and the recess are disposed with angular differences of 90 degrees. The series of the link elements is bendable in the two lateral directions, but is kept rigid in the longitudinal direction and rotational direction of twist. In a manner similar to the link elements of the steering unit, the link elements are tubular circularly with the guide lumen at the center for penetration of the medical instrument. The control wire extends through the guide lumen in the link elements.

In FIG. 35A, the flexible section of U.S. Pat. No. 7,637,905 is illustrated, and includes a control wire 100 or pull wire and link elements 101. The control wire 100 passes a position offset radially from an axis 102 which extends crosswise to the pivot axis of the link elements 101. If the steering unit is bent for steering by pull of the control wire 100, the flexible section 103 becomes bent by orienting the control wire 100 inwards, as illustrate in FIG. 35B. Even if a doctor or operator wishes to bend only the steering unit, the flexible section 103 is likely to bend, and may push the overtube radially from a projecting portion of the flexible section 103. Problems arise in that the overtube may be curved in spite of an intended straight form, or may extend straight in spite of an intended curved form. Easy handling is impossible, because the flexible section 103 must be adjusted for restart of manipulation.

### SUMMARY OF THE INVENTION

In view of the foregoing problems, an object of the present invention is to provide a torque transmission device in which torque can be transmitted with exactitude by removing unwanted influence of tension applied to a control wire.

In order to achieve the above and other objects and advantages of this invention, a torque transmission device extending in a longitudinal axis direction is provided, and includes a first link element having a first guide lumen. A second link element is disposed at a distal end of the first link element in the longitudinal axis direction, and has a second guide lumen. A control wire is disposed through the first and second guide lumens movably in the longitudinal axis direction. A first pivot mechanism couples the first link element to the second link element in a rotatable manner about a first pivot axis being perpendicular to the longitudinal axis direction. The control wire through the first and second guide lumens extends in a coplanar manner with the first pivot axis and crosswise thereto.

The first pivot mechanism includes a first front connecting portion positioned on the first link element. A first rear connecting portion, positioned on the second link element, and engaged with the first front connecting portion in a rotatable manner.

Furthermore, a third link element is disposed at a distal end of the second link element in the longitudinal axis direction, has a third guide lumen, for receiving insertion of the control wire movably in the longitudinal axis direction. A fourth link element is disposed at a distal end of the third link element in the longitudinal axis direction, has a fourth guide lumen, for receiving insertion of the control wire movably in the longitudinal axis direction. A second pivot mechanism couples the third link element to the fourth link element in a rotatable manner about a second pivot axis being perpendicular to the longitudinal axis direction. The control wire through the third and fourth guide lumens extends in a non-coplanar manner with the second pivot axis and crosswise thereto.

The second pivot mechanism includes a second front connecting portion positioned on the third link element. A second rear connecting portion is positioned on the fourth link element, and engaged with the second front connecting portion in a rotatable manner.

Furthermore, a distal link element is positioned in a distal end of a link group including the first to fourth link elements, and provided with a distal end of the control wire retained thereon. A handle section is secured to a proximal end of the link group, for applying torque to the link group when rotated about an axis extending in the longitudinal axis direction. A wire puller is disposed on the handle section, secured to a proximal end of the control wire, for pulling the control wire when operated externally.

Furthermore, a fifth link element is disposed at a proximal end of the first link element in the longitudinal axis direction, has a guide lumen, for receiving insertion of the control wire movably in the longitudinal axis direction. An additional pivot mechanism couples the fifth link element to the first link element in a rotatable manner about a predetermined pivot axis being perpendicular to the longitudinal axis direction. The predetermined pivot axis extends in a non-coplanar manner with the first pivot axis and crosswise thereto.

The additional pivot mechanism includes an additional front connecting portion positioned on the fifth link element. An additional rear connecting portion is positioned on the first link element, and engaged with the additional front connecting portion in a rotatable manner.

There is a flexible section having the first and second link elements. A steering unit has the third and fourth link elements.

Furthermore, an intermediate link element connects the second link element with the third link element. A third pivot mechanism couples the intermediate link element to the second link element in a rotatable manner about a third pivot axis extending crosswise to the control wire in a coplanar manner. A fourth pivot mechanism couples the intermediate link element to the third link element in a rotatable manner about a fourth pivot axis extending in a non-coplanar manner with the control wire and crosswise thereto.

The first pivot mechanism further includes a contact surface, formed on the first or second link element in a retracted manner, for allowing the first and second link elements to rotate relative to one another, the contact surface having a predetermined width in a first direction perpendicular to the first pivot axis, the predetermined width being equal to or more than a width of the first and second guide lumens in the first direction.

Furthermore, a coating is applied to the contact surface, for increasing resistance of friction.

Furthermore, a fine surface pattern of projections or recesses, formed on the contact surface, for increasing resistance of friction.

The contact surface includes an arcuate surface. A flat surface is formed by partially chamfering the arcuate surface.

Furthermore, a coating is applied to the contact surface, for increasing slipping property.

The first pivot mechanism includes a connecting projection being arcuate when viewed in a section. A connecting recess is arcuate when viewed in a section, for receiving the connecting projection. An inclined surface is formed with each of two walls of the connecting recess, for guiding the connecting projection toward the connecting recess.

The first pivot mechanism includes a connecting projection being arcuate when viewed in a section. A connecting recess is arcuate when viewed in a section, for receiving the connecting projection. A snap-fit structure allows push of the connecting projection into the connecting recess by resilient deformation for assembly, and keeps the connecting projection rotatable in the connecting recess after the push.

Also, the torque transmission device is used with an endoscope and entered in a body cavity together with an elongated tube of the endoscope.

In one preferred embodiment, the torque transmission device is incorporated in a high frequency snare instrument for medical use.

Consequently, torque can be transmitted with exactitude by removing unwanted influence of tension applied to a control wire, because the control wire through the guide lumens in the link elements is set coplanar with the first pivot axis of the link elements.

### BRIEF DESCRIPTION OF THE DRAWINGS

The above objects and advantages of the present invention will become more apparent from the following detailed description when read in connection with the accompanying drawings, in which:

FIG. 1 is a perspective view, partially broken, illustrating a device;

FIG. 2 is a side elevation illustrating a steering unit of the device;

FIG. 3 is a perspective view illustrating link elements in the steering unit;

FIGS. 4A-4D are elevations and a plan illustrating each one of link elements;

FIG. 5 is a vertical section illustrating the link element;

FIG. 6 is a vertical section illustrating a link group of the link elements;

FIG. 7 is a vertical section illustrating bend of the link group;

FIG. 8 is a side elevation illustrating a link group in a flexible section;

FIGS. 9A and 9B are perspective views illustrating a link element included in the flexible section;

FIGS. 10A-10D are elevations and plans illustrating the link element in the flexible section;

FIG. 11 is a vertical section illustrating the link element;

FIG. 12 is a vertical section illustrating the link element;

FIG. 13 is a vertical section illustrating a relationship between widths A and C;

FIG. 14 is a vertical section illustrating a handle section of a torque transmission device;

FIG. 15 is a perspective view, partially broken, illustrating manipulation with the torque transmission device for transluminal endoscopic surgery;

FIG. 16 is a cross section illustrating a state of entry of a tube of an endoscope and that of the torque transmission device into an overtube;

FIG. 17 is an explanatory view illustrating protrusion of a steering unit from the overtube;

FIG. 18 is an explanatory view illustrating bending of the overtube along the bend of the steering unit;

FIG. 19 is an explanatory view illustrating bending of the overtube along the bend of a steering unit of the endoscope;

FIG. 20 is an explanatory view illustrating a state of approach of the endoscope to an object in a body cavity;

FIG. 21 is a vertical section, partially broken, illustrating one preferred link element having a fine surface pattern for high friction;

FIGS. 22A and 22B are explanatory views illustrating another preferred link element having a flat contact surface;

FIG. 23 is a cross section illustrating one preferred link element having a double sleeve structure;

FIG. 24 is a cross section illustrating another preferred link element having four supports;

FIG. 25 is a cross section illustrating still another preferred link element having a hexagonal guide lumen;

FIG. 26 is a perspective view, partially broken, illustrating another preferred link element having inclined surfaces;

FIG. 27 is a vertical section, partially broken, illustrating still another preferred link element having a snap-fit structure;

FIG. 28 is a side elevation illustrating another preferred combination of link groups in a steering unit and a flexible section;

FIGS. 29A and 29B are side elevations illustrating a preferred use of a torque transmission device in a high frequency snare instrument;

FIG. 30 is a vertical section, partially broken, illustrating a handle section of the high frequency snare instrument;

FIG. 31 is an explanatory view illustrating a distal portion of the high frequency snare instrument with a distal link element;

FIGS. 32A and 32B are side elevations illustrating another preferred use of a torque transmission device in a high frequency snare instrument;

FIG. 33 is a vertical section, partially broken, illustrating a handle section of the high frequency snare instrument;

FIG. 34 is an explanatory view illustrating a distal portion of the high frequency snare instrument with a distal link element;

FIGS. 35A and 35B are side elevations, partially broken, illustrating a link group of link elements in a flexible section according to the prior art.

### DETAILED DESCRIPTION OF THE PREFERRED EMBODIMENT(S) OF THE PRESENT INVENTION

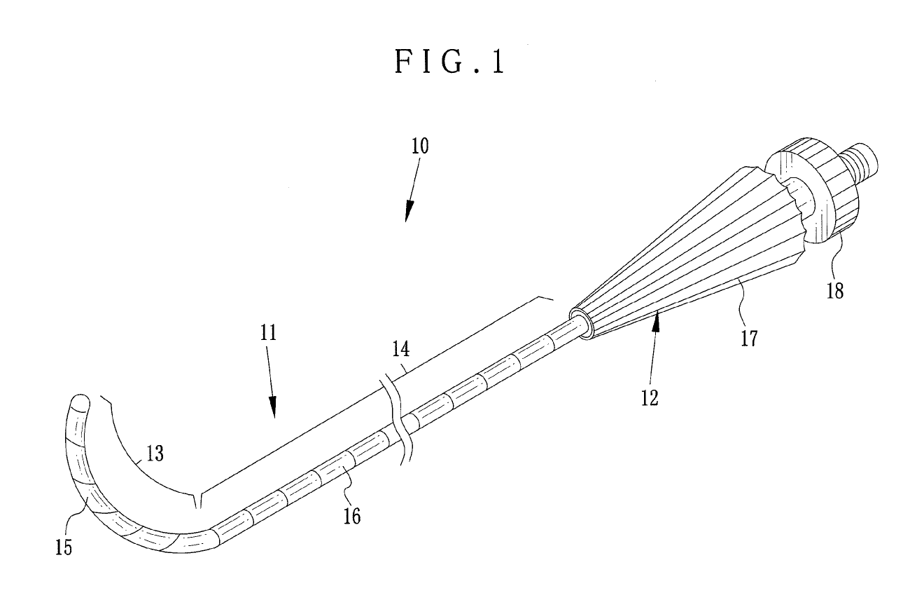

In FIG. 1, a torque transmission device 10 or torque transmission assembly is illustrated. The torque transmission device 10 includes a guide tube structure 11 or insertion tube of an elongated form, and a handle section 12. The guide tube structure 11 is introduced in an overtube or an instrument channel of an endoscope. The handle section 12 is positioned on a proximal side of the guide tube structure 11. The guide tube structure 11 includes a steering unit 13 and a flexible section 14 disposed on a proximal side of the steering unit 13.

A plurality of link elements or sleeves 15 for the steering unit (third and fourth link elements) are connected with one another to constitute the steering unit 13. A link elements or sleeves 16 for the flexible section (first, second and fifth link elements) are connected with one another to constitute the flexible section 14. Only one control wire is disposed to extend through the guide tube structure 11. Examples of materials for forming the link elements 15 and 16 may be resin, metal and other suitable substances having sufficient rigidity. Among those, metal is specifically preferable if a decrease in the diameter of the guide tube structure 11 is desired for some purposes.

A grip 17 and a rotatable wheel 18 are disposed on the handle section 12. The wheel 18 is rotatable about an axis of the guide tube structure 11 extending in a longitudinal axis direction. When the wheel 18 is rotated in one direction, one control wire is pulled to bend the steering unit 13 in one direction for steering. When the wheel 18 is rotated back to its original position, the steering unit 13 is bent back and returns to its straight form. Also, when the grip 17 is manually rotated with fingers of a user, torque of the grip 17 is transmitted by the flexible section 14 to the steering unit 13. Thus, the steering unit 13 can be oriented in a desired direction by adjustment.

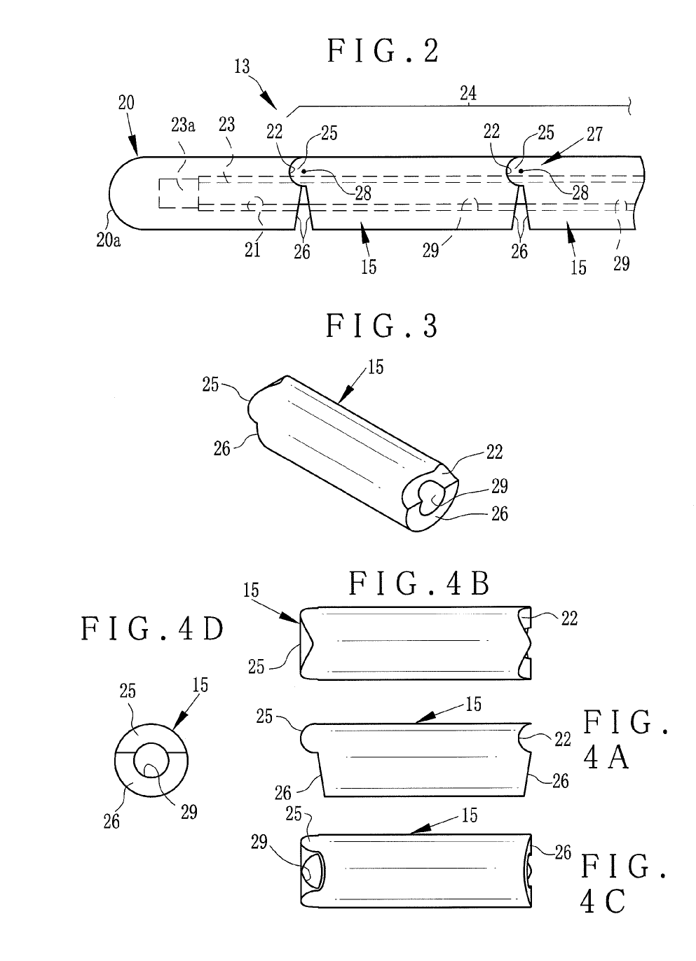

In FIG. 2, a distal link element 20 is combined with a plurality of the link elements 15 to constitute the steering unit 13. A link end 20a of the distal link element 20 is hemispherical. A hole 21 and a connecting recess 22 as a rear connecting portion are formed in a proximal end of the distal link element 20. A control wire 23 is entered in the hole 21. In the proximal end, the hole 21 is located on an axis of the distal link element 20 extending in the longitudinal axis direction. A distal wire end 23a is fixedly retained on a bottom surface of the hole 21 upon entry of the control wire 23 in the hole 21.

The connecting recess 22 of the distal link element 20 is shaped semi-circularly as viewed in a section. A pivot axis of the connecting recess 22 is perpendicular to the longitudinal axis direction of the link elements. A position of the connecting recess 22 is eccentric with respect to the longitudinal axis direction, and at one point of a tubular end surface of the distal link element 20. There is a link group 24 or multi link component for the steering unit. A connecting projection 25 as a front connecting portion is formed with a distal one of the link elements 15 in the link group 24. The connecting recess 22 is engaged with the connecting projection 25. An inclined surface 26 is formed by chamfering a portion of the end surface other than the connecting recess 22, and defines a gap for bending in cooperation with an adjacent one of the link elements 15.

Each one of the link elements 15 is a sleeve, has a first end where the connecting projection 25 and the inclined surface 26 are formed, and a second end where the connecting recess 22 and the inclined surface 26 are formed. See FIGS. 3, 4A, 4B, 4C, 4D and 5. A first pivot mechanism 27 for articulation is constituted by a combination of the connecting projection 25 and the connecting recess 22 engaged together. The connecting recess 22 has the same shape as that of the distal link element 20. The connecting projection 25 has a shape of an arc of a semi-circle. A pivot axis of the connecting projection 25 is perpendicular to the longitudinal axis direction of the link elements. A position of the connecting projection 25 is eccentric with respect to the longitudinal axis direction, and at one point of a tubular end surface of the link elements 15. A pivot axis 28 for articulation of FIG. 2 is defined by the connecting projection 25 and the connecting recess 22 of the link elements 15 for bending the steering unit 13 in one direction. Each link element in the link group 24 with the distal link element 20 rotationally moves about the pivot axis 28 which is positioned eccentrically from the longitudinal axis of the link elements.

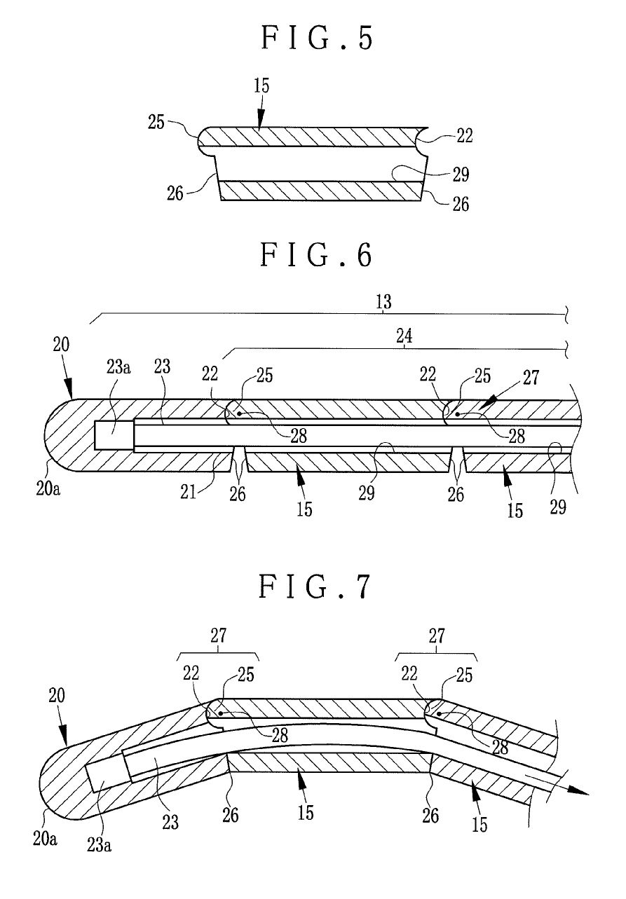

In FIGS. 4A, 4B, 4C, 4D and 5, the form of one of the link elements 15 is depicted. FIG. 4A is a front elevation. FIG. 4B is a top plan. FIG. 4C is a bottom plan. FIG. 4D is a left side elevation. The link element 15, when viewed in a right side elevation, appears in the same manner as FIG. 4D, and when viewed in a rear elevation, appears symmetrically with respect to FIG. 4A.

The link element 15 is shaped circularly as viewed in a section. In FIG. 6, a guide lumen 29 or wire lumen as guide portion is formed through the link element 15 axially for passing the control wire 23. The guide lumen 29 keeps the control wire 23 positioned on the axis. Also, the guide lumen 29 sets the control wire 23 on a line non-coplanar with the pivot axis 28 of the first pivot mechanism 27, namely in a condition of non-intersecting skew lines being perpendicularly crossed when viewed in a projected manner. In FIG. 7, when the control wire 23 is pulled, the steering unit 13 is bent by swing about the pivot axis 28 between the connecting recess 22 and the connecting projection 25 in a direction to narrow an interval between the link elements 15 and an interval between the distal link element 20 and the link elements 15, or a direction to orient the inclined surface 26 internally on a side opposite to the connecting recess 22 and the connecting projection 25. The steering unit 13 is bendable to an extent of contact of two of the inclined surfaces 26 on the link elements 15 and the distal link element 20. The connecting projection 25 and the connecting recess 22 constitute the front connecting portions of the invention.

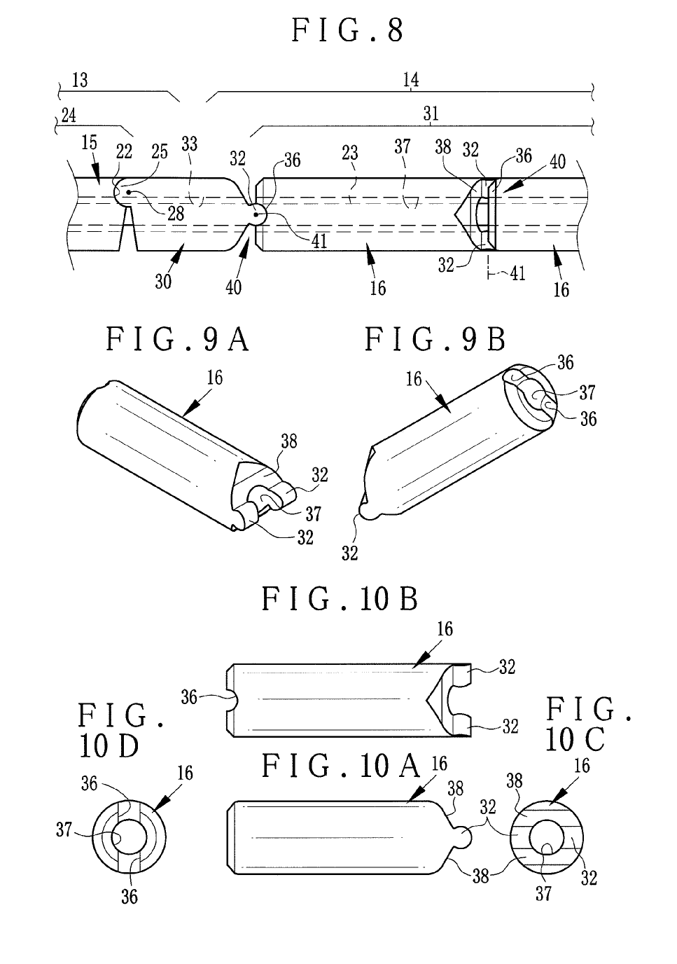

In FIG. 8, an intermediate link element 30 or sleeve is connected to a proximal end of the link group 24. A link group 31 or multi link component constitutes the flexible section 14. The intermediate link element 30 connects the link group 24 with the link group 31. The intermediate link element 30 has first and second ends. The connecting projection 25 is formed on the first end for engagement with the connecting recess 22 of the link elements 15. Connecting projections 32 as a rear connecting portion are formed on the second end. A guide lumen 33 or wire lumen is formed through the intermediate link element 30 for receiving penetration of the control wire 23 along the longitudinal axis. The connecting projection 25 is structurally the same as that of the link elements 15. The connecting projections 32 are two portions between which the guide lumen 33 is located, and are formed in an arcuate shape as viewed in a section and symmetrically with respect to the longitudinal axis direction. The connecting projections 32 of the intermediate link element 30 are structurally the same as those of the link elements 16 to be described later for the purpose of connection of the link group 31. Note that in the first pivot mechanism 27, the connecting recess 22 is located in the distal direction and the connecting projection 25 is located in the proximal direction. However, it is possible to determine the directions of locating the connecting recess 22 and the connecting projection 25 in a manner opposite to those of the embodiment.

The link group 31 is a series of the link elements 16. Each of the link elements 16 has connecting recesses 36 as a front connecting portion, inclined surfaces 38, and a guide lumen 37 or wire lumen. The connecting recesses 36 are located at a first end of the link elements 16. The inclined surfaces 38 and the connecting projections 32 are located at a second end of the link elements 16. The guide lumen 37 is formed to extend in the longitudinal axis direction. See FIGS. 9A, 9B, 10A, 10B, 10C, 10D, 11 and 12. A second pivot mechanism 40 for articulation is defined with a pivot axis 41, and constituted by the connecting projections 32 and the connecting recesses 36 for connection between the link elements 16. The guide lumen 37 keeps the control wire 23 movable so that the pivot axis 41 intersects with the control wire 23 in a coplanar manner. The connecting projections 32 are formed arcuately and symmetric with one another with respect to the longitudinal axis direction or the guide lumen 37 at an end surface of the link elements 16. The inclined surfaces 38 operate in the same manner as the inclined surface 26, and are formed with sides between which the connecting projections 32 are disposed.

The connecting recesses 36 are formed arcuately as viewed in a section, and symmetrically with respect to the longitudinal axis direction, and so disposed that the guide lumen 37 is positioned between those. The connecting recesses 36 at a first end of the link elements 16 and the connecting projections 32 at a second end of the link elements 16 are so constructed that the pivot axis 41 of a distal side is kept non-coplanar with the pivot axis 41 of a proximal side, namely in a condition of non-intersecting skew lines being perpendicularly crossed when viewed in a projected manner. In short, the pivot axis 41 of the connecting recesses 36 is directed to extend with an angular difference of 90 degrees from the pivot axis 41 of the connecting projections 32. As the connecting projections 32 are engaged with the connecting recesses 36 with differences in the angle of the pivot axis 41 at 90 degrees, a bendable property of the link elements 16 is given in the four directions. The link elements 16 can have rigidity with respect to its longitudinal direction and rotational direction of twist. Note that in the second pivot mechanism 40, the connecting recesses 36 are oriented in the distal direction and the connecting projections 32 are oriented in the proximal direction. However, it is possible to determine the directions of the connecting recesses 36 and the connecting projections 32 in a manner opposite to those of the embodiment. The connecting projections 32 or the connecting recesses 36 constitute a front connecting portion as a feature of the invention.

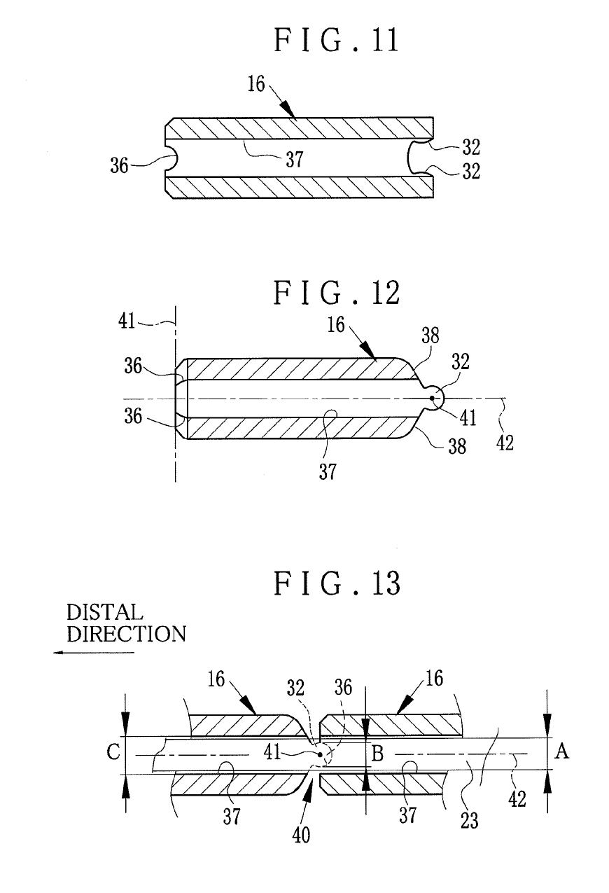

In FIGS. 10A, 10B, 10C, 10D and 11, the form of one of the link elements 16 is depicted. FIG. 10A is a front elevation. FIG. 10B is a top plan. FIG. 100 is a right side elevation. FIG. 10D is a left side elevation. The link element 16, when viewed in a bottom plan, appears in the same manner as FIG. 10B, and when viewed in a rear elevation, appears symmetrically with respect to FIG. 10A.

In FIG. 12, a longitudinal axis 42 or a longitudinal axis direction is defined to extend through the guide lumen 37. The control wire 23 is guided by the guide lumen 37 on the longitudinal axis 42 which intersects with the pivot axis 41 defined by the connecting projections 32 and the connecting recesses 36 of the second pivot mechanism 40. Therefore, even when the steering unit 13 is bent by pull of the control wire 23, the link group 31 does not bend but can maintain its shape.

Note that it is unnecessary for the guide lumen 37 to maintain the control wire 23 on the longitudinal axis 42 with high exactitude. See FIG. 13. The guide lumen 37 can have such an inner diameter C for guiding that a width of the control wire 23 is enough to locate the control wire 23 within a range of a width B of the connecting projections 32 and the connecting recesses 36 (C-A<B) even if the control wire 23 incidentally moves tortuously up and down as viewed laterally in the drawing. Note that a condition of C<B is preferable in which the diameter of the arc of the connecting projections 32 and the connecting recesses 36 is enlarged to enlarge the width B of the connecting projections 32 and the connecting recesses 36 over the inner diameter C of the guide lumen 37. This is because the curved form of the link group 31 will not receive influence of tension of the control wire 23.

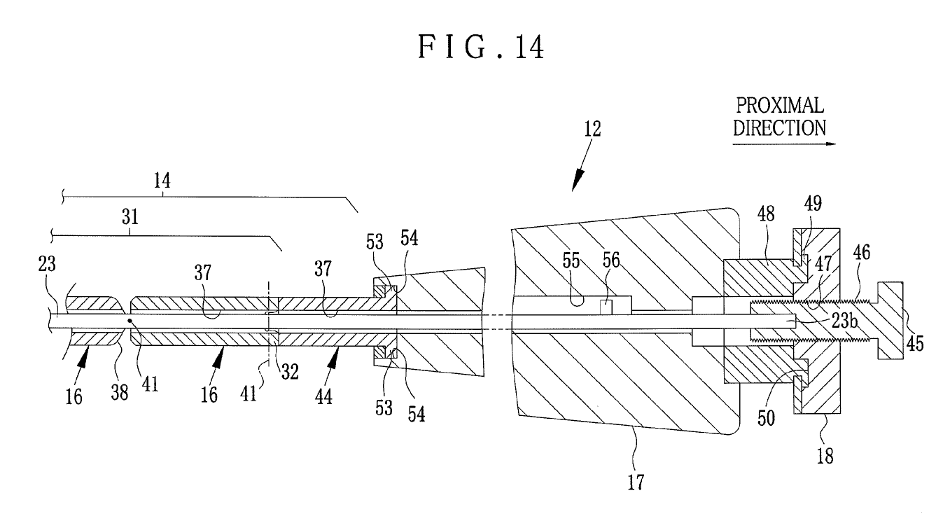

In FIG. 14, a proximal link element 44 or sleeve for the flexible section at a proximal end of the link group 31 is secured firmly to the grip 17. A wheel support 48 or cuff is secured firmly to a proximal end of the grip 17. A flange 49 is disposed to project from a proximal end of the wheel support 48. An annular groove 50 is formed in the wheel 18, and engaged with the flange 49. The wheel 18 is rotatable relative to the flange 49.

Female threads 47 are formed on an inner surface of the wheel 18 as screw hole. An end tab portion 45 has male threads 46, which are engaged helically with the female threads 47. A proximal wire end 23b of the control wire 23 is fixedly secured to the end tab portion 45.

A wire puller including the wheel 18 also has a mechanism for preventing twist of the control wire 23. The mechanism includes a key groove 55 and a key projection 56. The key groove 55 is formed in an inner surface of the grip 17 and extends longitudinally. The key projection 56 is formed on the control wire 23. The key projection 56 is engaged with the key groove 55 to prevent the control wire 23 from twisting. The end tab portion 45 can be kept fixed rotationally.

When the wheel 18 is rotated in a first direction, the end tab portion 45 is moved in a proximal direction on the axis. The control wire 23 is pulled to bend the steering unit 13 in one direction for steering. When the wheel 18 is rotated in a second direction, the end tab portion 45 is moved in a distal direction on the axis. The control wire 23 is loosened to release the steering unit 13 from being bent for steering.

A pair of key projections 53 are formed on sides of the proximal link element 44 at the proximal end, so that the guide lumen 37 is located between those. A key groove 54 is formed inside an end portion of the grip 17, and is engaged with each of the key projections 53. When the grip 17 is rotated, its torque is transmitted by the flexible section 14 to the steering unit 13, of which a direction can be adjusted as desired by an operator.

Note that the control wire is single, but can operate for steering in one intended direction. At first, the handle section is rotated about the longitudinal axis to determine an orientation of the steering unit. Then the wheel of the wire puller is rotated so that the bending of the steering unit in the determined orientation is possible.

Various known mechanisms can be used for the wire puller. For example, a mechanism including a pull lever for pulling the control wire 23 can be used. The pull lever has a first end where the control wire 23 is secured and a second end disposed to protrude externally. A rotational axis of the pull lever is disposed between the first and second ends. When the pull lever is swung in a distal direction, the control wire 23 is pulled. A lock mechanism for locking the pull lever in the swing position is disposed. When the lock mechanism is released, the pull lever comes back to an erect position, to loosen the control wire 23.

The torque transmission device 10 is usable for pressing an organ in a body with considerable softness to ensure a large field of view for surgery as described with the prior art, and also for guiding an overtube to reach an object of interest.

For example, the Natural Orifice Transluminal Endoscopic Surgery (NOTES) is known, in which a gastric submucosal tumor or pancreas is treated by entry into a abdominal cavity through a lumen of a large intestine, rectum, vagina or the like. This method has been recently highlighted owing to small physical stress to a body of a patient in comparison with abdominal surgery or laparoscopic surgery. For example, the endoscope enters the abdominal cavity through the vagina. The sacral spine is located on a straight line passing through the affected tissue and a point of entry. It is necessary for the overtube to reach the stomach or pancreas by steering the overtube in an S shape away from the sacral spine.

Examples of the overtube are disclosed in U.S. Pat. No. 5,174,276 (corresponding to JP-A 4-501676) and U.S. Pat. No. 5,337,733 (corresponding to JP-A 5-503434), in which its condition is changeable between flexible and rigid states even during the use. The overtube, when in the flexible state, is freely flexible, and when in the rigid state, is prevented from flexing. An example of endoscope is an electronic endoscope, of which an elongated tube or insertion tube is constituted by a head assembly, a steering unit and a flexible section arranged in a proximal direction. The steering unit is bendable in a direction as desired by operating the handle section. The flexible section extends between the steering unit and the handle section. An image pickup device is incorporated in the head assembly. An image is produced by the endoscope, displayed on a monitor display panel, and observed by a doctor or operator who manipulates the overtube for surgery.

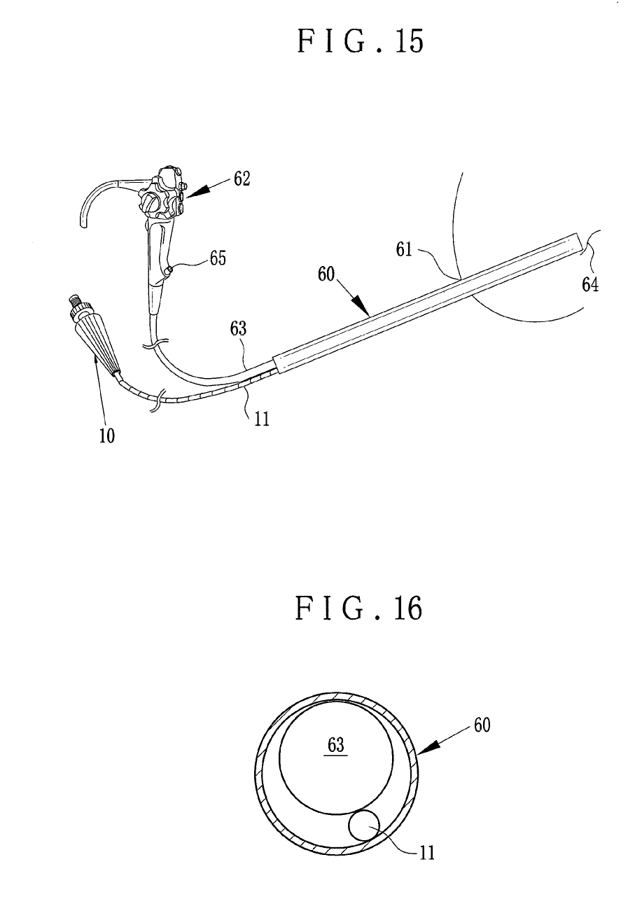

In FIGS. 15 and 16, a state of the Natural Orifice Transluminal Endoscopic Surgery (NOTES) is illustrated. At first, an overtube 60 or jacket tube is entered in a vagina 61. Then an elongated tube 63 or insertion tube of an endoscope 62 for colposcopy or probe is introduced in the overtube 60. Also, the guide tube structure 11 of the torque transmission device 10 is introduced in the overtube 60. A surgical instrument is inserted in an instrument channel 65 of the endoscope 62, to incise the posterior vaginal formix, for the overtube 60 to reach a site short of a sacral spine 64. Note that the surgical instrument is not depicted for simplifying the depiction.

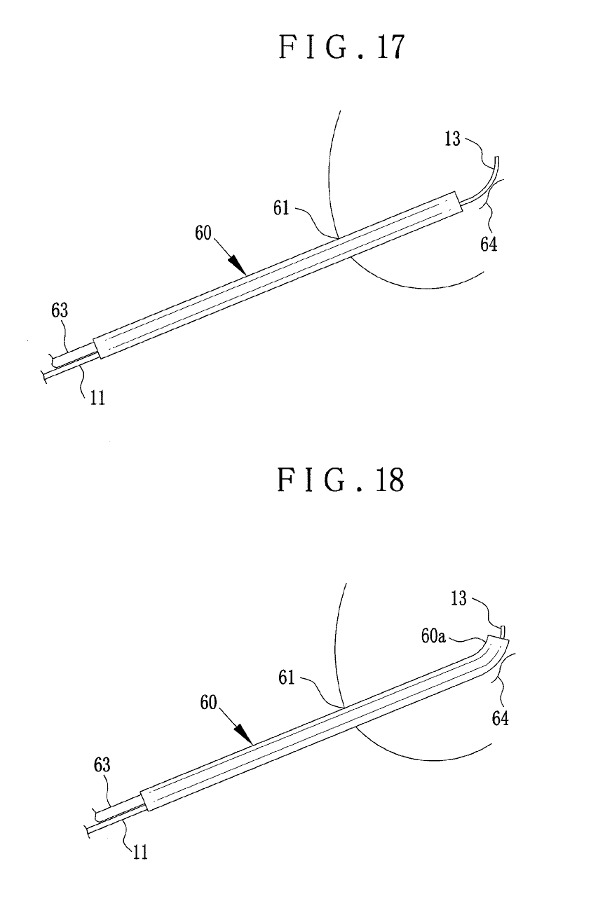

In FIG. 17, the overtube 60 is released from retention on the torque transmission device 10. The steering unit 13 of the torque transmission device 10 is moved to protrude from the overtube 60 at a predetermined amount, and is bent away from the sacral spine 64. Then the overtube 60 is advanced further. In FIG. 18, an overtube end 60a or jacket end of the overtube 60 becomes bent according to a curved form of the steering unit 13. Then the overtube 60 is anchored.

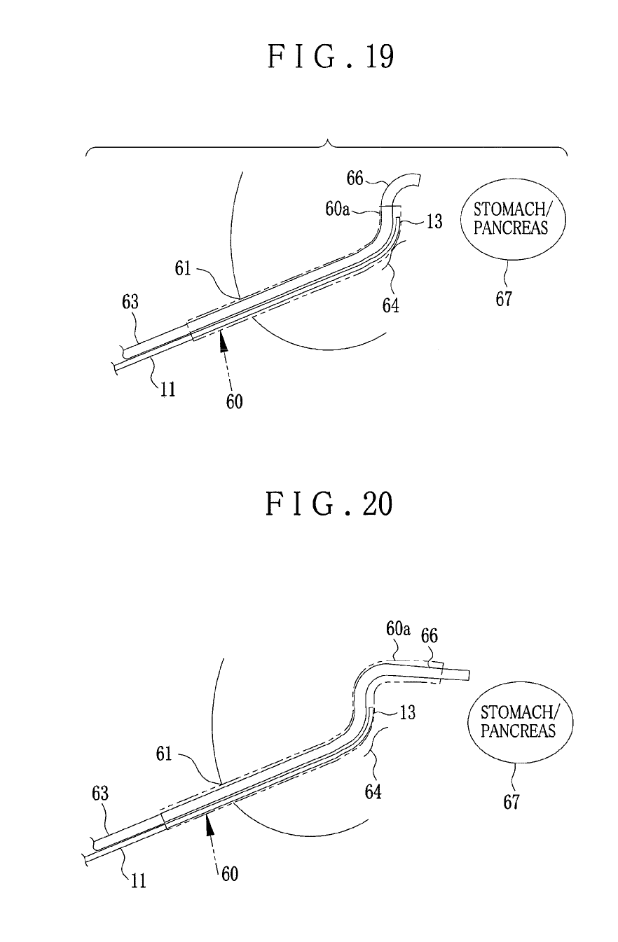

Then in FIG. 19, the elongated tube 63 of the endoscope 62 is entered. A steering unit 66 of the endoscope 62 is caused to protrude from the overtube 60, and bent in a direction opposite to the previous direction, namely a direction to set the distal end of the endoscope toward a stomach, pancreas or the like. Then the overtube 60 is released from locking, and is entered. In FIG. 20, the overtube end 60a of the overtube 60 becomes bent along the steering unit 66 of the endoscope 62. Then the overtube 60 is fixedly positioned. A stomach/pancreas 67 is approached by the elongated tube 63 of the endoscope 62 during advance. Thus, the overtube 60 can be bent in an S-shape by use of the steering of the steering unit 13 of the torque transmission device 10 and that of the steering unit 66.

In the torque transmission device 10, the flexible section 14 does not flex even upon bending the steering unit 13. An original form of the flexible section 14 is maintained. Thus, the torque transmission device 10 is effectively used for a purpose of bending the steering unit 13 for bypassing the sacral spine 64 and maintaining remaining portions without bending, specifically for the Natural Orifice Transluminal Endoscopic Surgery (NOTES).

It is possible to use various structures known in the art for the purpose of achieving the purpose of the invention.

There are various situations of no occurrence of influence to the flexed form of the flexible section 14 even in occurrence of tension with the control wire 23. In a first one of the situations, the tension with the control wire 23 engages the link elements 16 with one another to maintain the present shape of the flexible section 14. In a second one of the situations, the link elements 16 are movable from one another even in occurrence of the tension with the control wire 23, so that the flexible section 14 is still flexible.

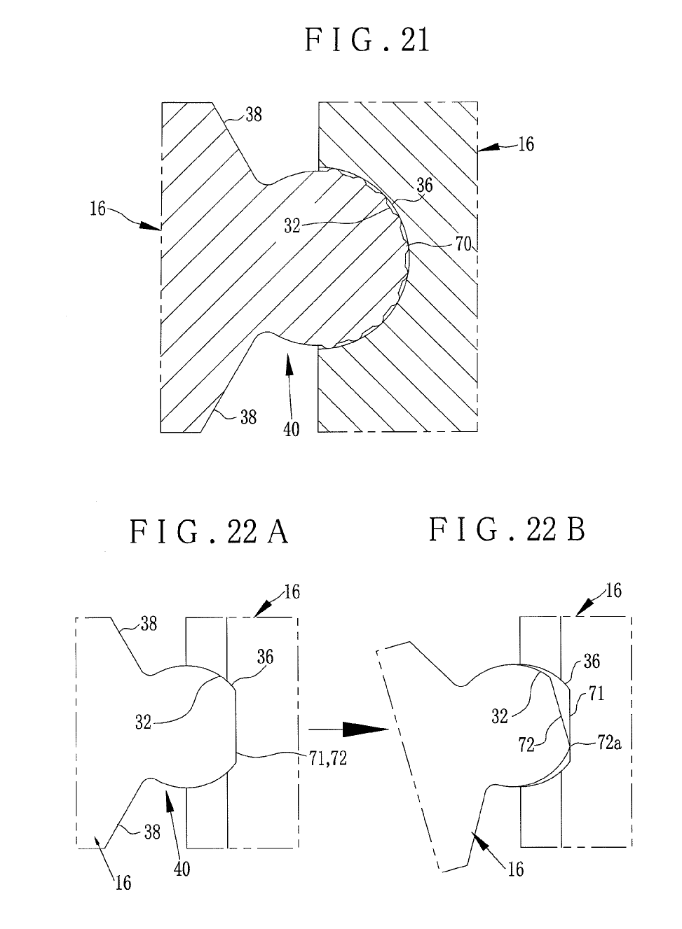

To engage the link elements 16 with one another for maintaining the shape in an unchanged manner, a coefficient of friction can be effectively increased between the connecting projections 25 and 32 and the connecting recesses 22 and 36 in the first and second pivot mechanisms 27 and 40. For example, a coating with the high coefficient of friction can be preferably applied to contact surfaces of the connecting projections 32 and the connecting recesses 36 in the second pivot mechanism 40. Also, fine projections 70 of a contact surface as a fine surface pattern can be formed on the connecting projections 32, the connecting recesses 36 or on both of those in place of the coating. See FIG. 21. Also, in FIG. 22A, it is possible to form flat surfaces 71 and 72 by partially chamfering contact surfaces of the connecting projections 32 and the connecting recesses 36. In FIG. 22B, the link element 16 rotates. An edge 72a of the one flat surface 72 contacts the flat surface 71 to increase the coefficient of friction. It is of course possible to utilize the construction of FIGS. 21 and 22 for the first pivot mechanism 27.

Furthermore, it is preferable to apply a coating of diamond-like carbon (DLC) to surfaces of the connecting projections 32 and the connecting recesses 36 specifically for the purpose of maintaining slide between the link elements 16 and flexibility in the flexible section 14 even upon occurrence of tension in the control wire 23, because the diamond-like carbon (DLC) has slipping property and resistance to abrasion. The coating is effective in lowering a friction coefficient of the surfaces and increasing the slipping property.

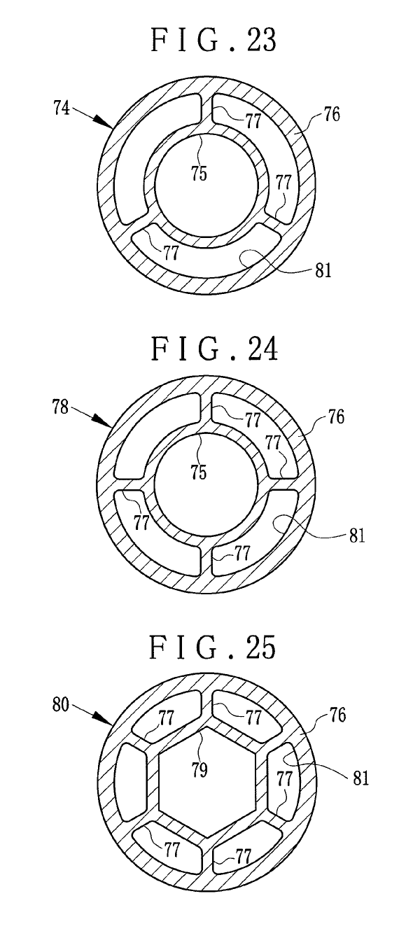

The link elements 16 of the above embodiment have the guide lumen 37 extending in the longitudinal axis direction. Furthermore, the link elements 16 may be formed in a double sleeve structure. For this purpose, a link element includes an inner barrel and an outer barrel. In FIG. 23, a link element 74 or sleeve for the flexible section is illustrated. There is an inner barrel and an outer barrel 76. A guide lumen 75 or wire lumen is defined in the inner barrel. Three support walls 77 are formed between the inner barrel and the outer barrel 76, and arranged radially. This is effective in reducing weight of the guide lumen 37. The number of the support walls 77 may be two or four or more. In FIG. 24, a link element 78 or sleeve for the flexible section has four of the support walls 77. In any of the structures of FIGS. 23 and 24, the support walls 77 are preferably disposed symmetrically on the periphery about the axis. In FIG. 25, one preferred link element 80 or sleeve for the flexible section is illustrated, in which a guide lumen 79 or wire lumen has a hexagonal shape as viewed in a cross section. Any suitable shape can be used, for example, circular shape, polygonal shape and the like for the guide lumen as viewed in a cross section. Thus, strength of the link element 80 can be high with the support walls 77. Note that lumens 81 defined between the outer barrel 76 and the wall of the guide lumen 75 or 79 can be utilized as an instrument channel for entry of a supply tube for supply of water or the like.

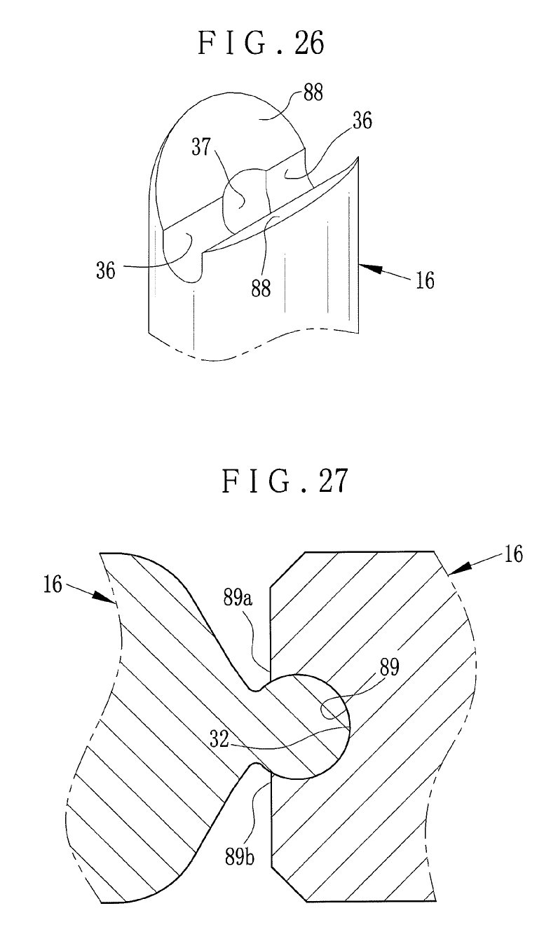

In FIG. 26, an additional structure for the connecting recesses 36 of the link elements 16 (See FIGS. 9A-12) is illustrated. Inclined surfaces 88 are formed with the connecting recesses 36, and arranged on its lateral sides for guiding. Even when the control wire 23 is loosened excessively to disengage the connecting recesses 36 from the connecting projections 32, the inclined surfaces 88 guide the connecting projections 32 to the connecting recesses 36 when the control wire 23 is pulled, to engage the connecting projections 32 with the connecting recesses 36.

In FIG. 27, a preferred mechanism for retaining with a click or snap-fit structure is illustrated. There are connecting holes 89 in which the connecting projections 32 are retained. Receiving walls 89a and 89b are a snap-fit structure of the connecting holes 89, are deformable with resiliency for receiving the connecting projections 32 over its maximum diameter. The receiving walls 89a and 89b are effective in tightening the engagement of the connecting projections 32 because over a half of the connecting projections 32 is retained by those to prevent easy disengagement.

In the above embodiments, the link group 24 for the steering unit is located at a distal end of the guide tube structure 11. The link group 31 for the flexible section is located at a proximal end of the guide tube structure 11. However, combinations of locations of the link groups 24 and 31 can be modified suitably for various purposes. It is possible to dispose a plurality of the link groups 24 at a predetermined distance in the guide tube structure 11. Also, the link group 31 may constitute a distal end portion, intermediate portion or proximal end portion of the guide tube structure 11.

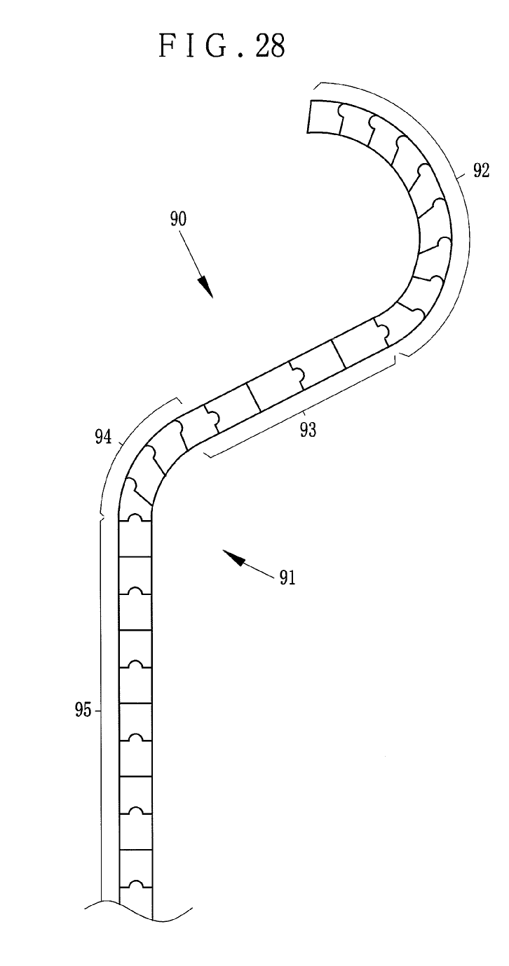

In FIG. 28, a guide tube structure 91 or insertion tube of an elongated form of a torque transmission device 90 or torque transmission assembly is illustrated. The guide tube structure 91 is a sequence of a link group 92 or multi link component for the steering unit, a link group 93 for the flexible section, a link group 94 for a steering unit, and a link group 95 for the flexible section. Each of the link groups 92 and 94 is bent for steering in one direction by pull of the control wire. The link groups 93 and 95 keep their shape at the time of the pull of the control wire irrespective of the pull. The guide tube structure 91 can be bent in an S shape by use of the link groups 92 and 94 disposed before and after the link group 93.

If the torque transmission device 10 or 90 of the above embodiments is flexed, acute edges may be formed at end portions of the link elements 15 and 16. It is likely that tissue in a body cavity is injured by the acute edges. Thus, a covering tube can be additionally used to cover the guide tube structure 11 of the torque transmission device 10 for the purpose of higher safety. It is possible only to cover the steering unit 13 with such a covering tube.

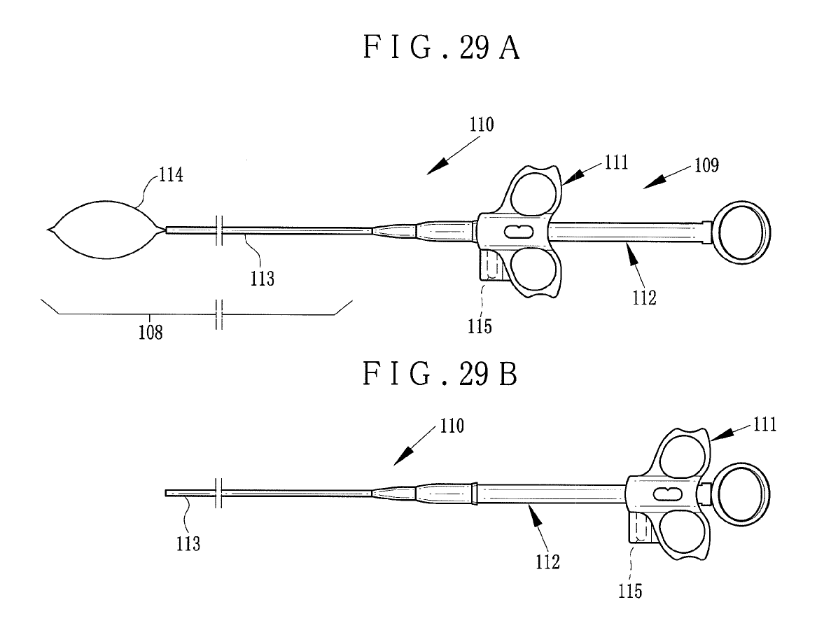

The torque transmission device of any of the above embodiments can be used with a medical instrument, for example, with a high frequency snare instrument 110. See FIGS. 29A and 29B. The high frequency snare instrument 110 includes an elongated tube 108 or insertion tube, and a handle section 109. The handle section 109 includes a slider 111 and a handle shaft 112. The elongated tube 108 includes a flexible sheath 113 and the link group 31. The flexible sheath 113 has a great length, and contains the link group 31. A snare loop 114 is movable back and forth relative to the flexible sheath 113. When the slider 111 is moved in the distal direction relative to the handle shaft 112, the snare loop 114 is moved to protrude from the flexible sheath 113 by movement of the link group 31. See FIG. 29A. When the slider 111 is moved in the proximal direction, the snare loop 114 is moved back to the inside of the flexible sheath 113. See FIG. 29B.

The snare loop 114, when advanced to protrude, becomes developed in a loop form with its resiliency, and when moved back into the flexible sheath 113, becomes squeezed by resilient deformation. An opening is formed partially in the slider 111. A plug 115 is disposed to appear in the opening for powering with high frequency current. The high frequency snare instrument 110 is entered in a body cavity through an instrument channel of an endoscope. The snare loop is moved to catch a root portion of protrusion of affected tissue, so as to excise polyp or the like by burning the polyp or firmly closing to cut the root portion.

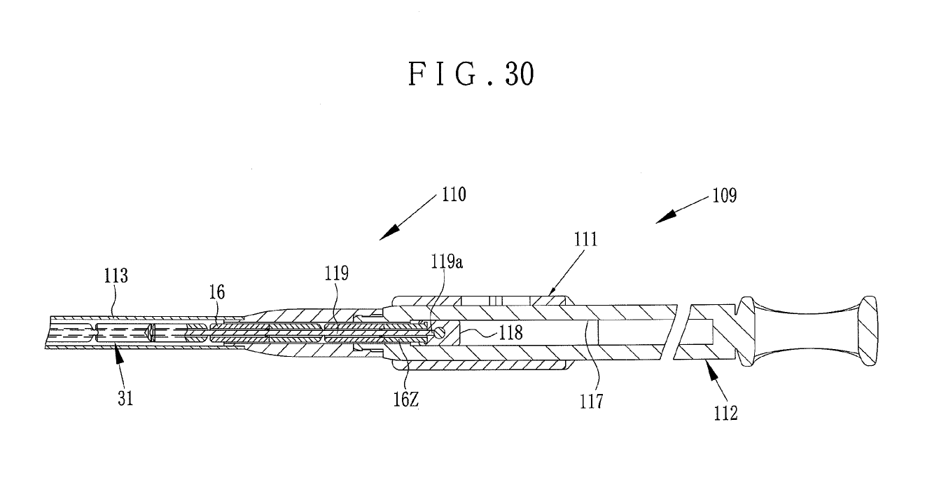

In FIG. 30, a slot 117 or groove is formed in the handle shaft 112 and extended in the longitudinal direction. A contact portion 118 on the slider 111 is engaged with the slot 117. A proximal wire end 119a of a control wire 119 is secured fixedly to the contact portion 118. Also, a proximal link element 16Z or sleeve is secured to the contact portion 118 on a proximal side in the link group 31. When the slider 111 is moved longitudinally, the link group 31 moves together with the control wire 119. The plug 115 is connected with the control wire 119. Each of the control wire 119 and the link elements 16 is formed from electrically conductive material, for example, stainless steel.

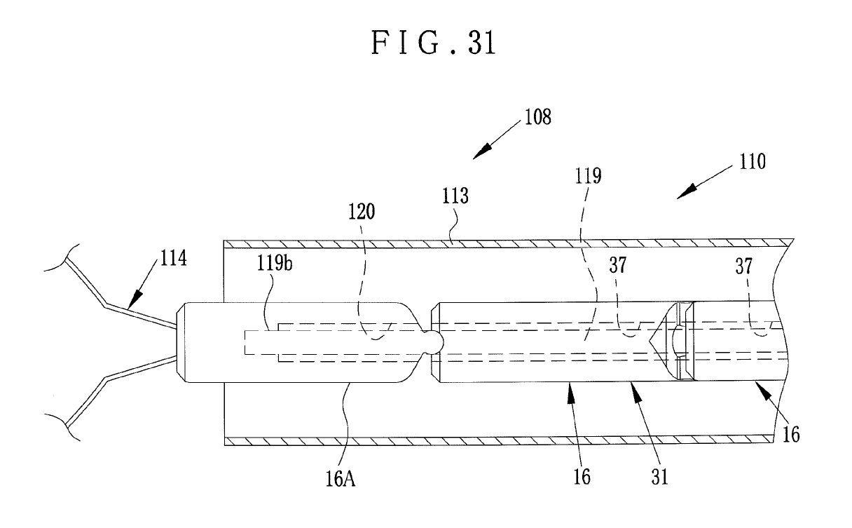

In FIG. 31, a distal link element 16A is positioned at a distal end of the link group 31. The snare loop 114 is secured to the distal link element 16A. A guide hole 120 or wire hole with a bottom portion is formed in the distal link element 16A. A distal wire end 119b of the control wire 119 is secured to the distal link element 16A inside the guide hole 120. High frequency current is caused to flow to the snare loop 114 from the control wire 119 and the link group 31. Only the link group 31 is disposed within the flexible sheath 113. The control wire 119 does not operate as operation wire for steering in the elongated tube 108. A distal end portion of the control wire 119 does not bend. The elongated tube 108 only operates for maintaining the flexibility. The control wire 119 keeps the link elements 16 engaged with one another. In operation, the handle section 109 is rotated so that the link group 31 transmits the rotation to the distal end portion. It is possible to set the snare loop 114 in a desired direction. Also, the link group 31 has flexibility and operates to maintain the flexibility.

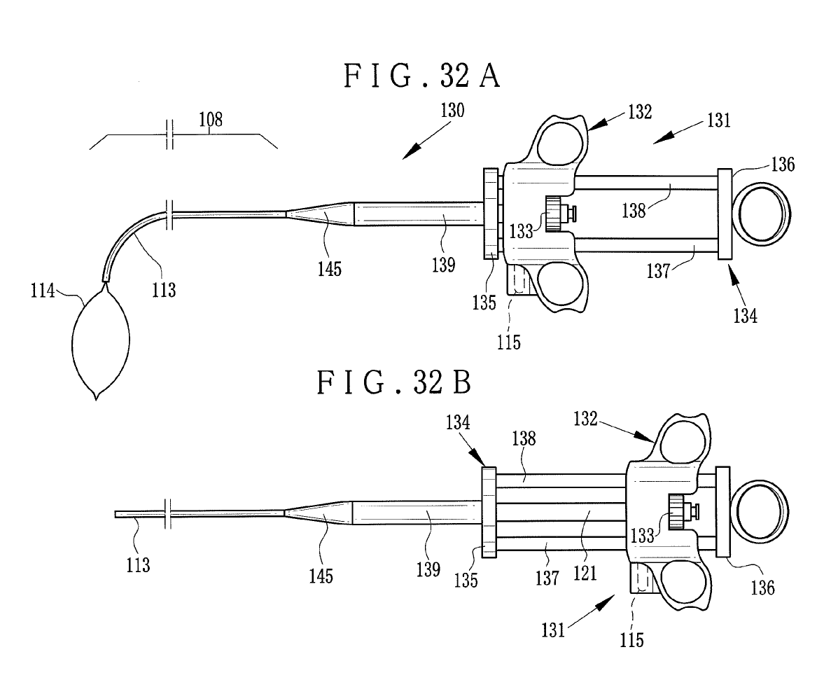

In FIGS. 32A and 32B, a high frequency snare instrument 130 by way of a torque transmission device is illustrated, in which a steering section is disposed at a distal end of the elongated tube 108. A handle section 131 is included in the high frequency snare instrument 130. A slider 132 is disposed on the handle section 131 in a slidable manner in the longitudinal direction. A rotatable wheel 133 is disposed on the slider 132 in a rotatable manner. A control wire (not shown) is pulled when the wheel 133 is rotated, to bend the steering section of the elongated tube 108. A support frame 134 or guide device includes a front support panel 135, a rear support panel 136 and a pair of guide rods 137 and 138, and has a quadrilateral form. The guide rods 137 and 138 are supported in parallel by the support panels 135 and 136 at a predetermined interval. The slider 132 is supported by the guide rods 137 and 138 in a slidable manner. The wheel 133 is disposed between the guide rods 137 and 138.

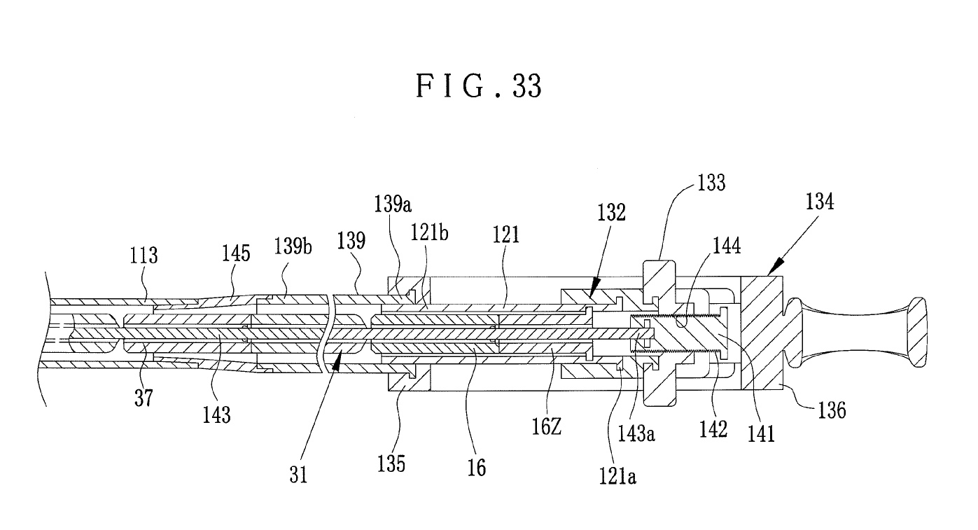

In FIG. 33, a rear barrel end 121a of an inner barrel 121 is secured to the slider 132 and positioned on the longitudinal axis. A front barrel end 121b of the inner barrel 121 is supported inside an outer barrel 139 in a slidable manner, so that the inner barrel 121 moves together with the slider 132. A rear barrel end 139a of the outer barrel 139 is secured to the front support panel 135. A front barrel end 139b of the outer barrel 139 is located at such a distance as to allow the inner barrel 121 to slide. A coupling 145 or connector for different diameters secures the flexible sheath 113 to the front barrel end 139b of the outer barrel 139. The proximal link element 16Z at a proximal end of the link group 31 is secured to the inner barrel 121 in a rotationally fixed manner. Female threads 144 are formed on an inner surface of the wheel 133. An end tab portion 141 has male threads 142, which is helically engaged with the female threads 144. A proximal wire end 143a of a control wire 143 is secured to the end tab portion 141. When the wheel 133 is rotated, the end tab portion 141 moves longitudinally according to the lead of the threads, to pull the control wire 143. The control wire 143 extends through the guide lumen 37 of the link elements 16. Such a wire puller can be the same as that depicted in FIG. 14. When the slider 132 is slid longitudinally, other elements move together, including the end tab portion 141, the control wire 143, the link group 31 and the inner barrel 121. Note that the plug 115 is connected with the control wire 143 by a connector which is not shown. Each of the control wire 143 and the link elements 16 is formed from electrically conductive material, for example, stainless steel.

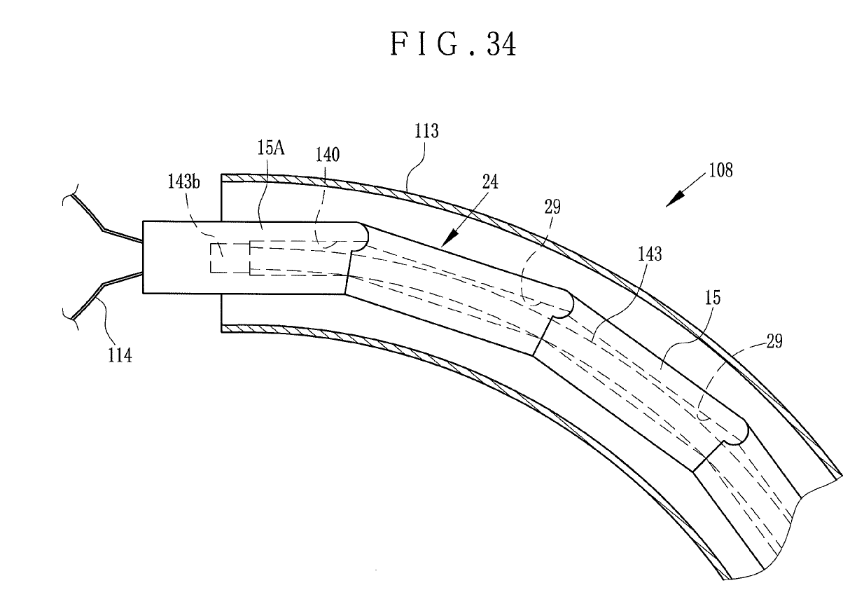

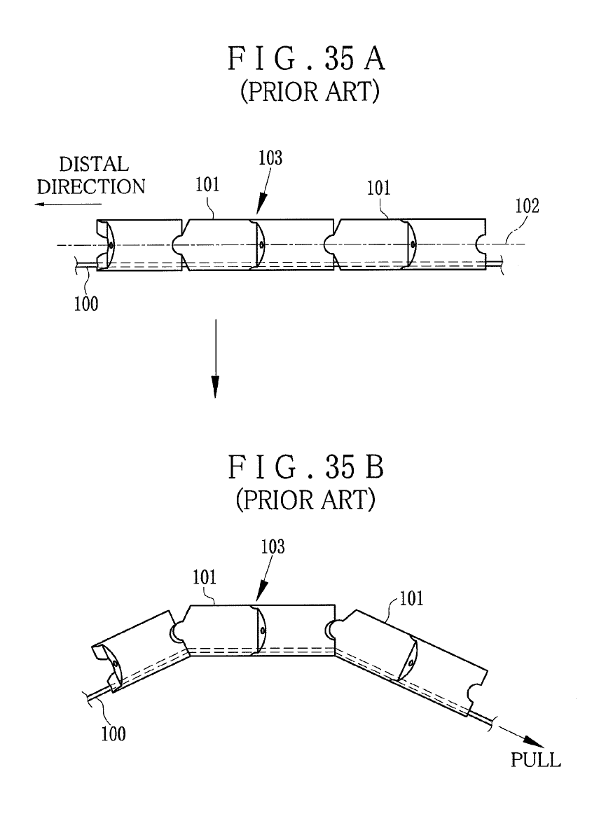

In FIG. 34, the link group 24 is connected with a distal end of the link group 31. The link elements 15 are formed from electrically conductive material, for example, stainless steel. A distal link element 15A is included in the link elements 15, and has a first end where the snare loop 114 is secured. A guide lumen 140 or wire lumen is formed in a second end of the distal link element 15A. The guide lumen 140 has a closed shape with a bottom. A distal wire end 143b of the control wire 143 is secured to the distal link element 15A through the guide lumen 140. A high frequency current is drawn through the control wire 143, the link groups 24 and 31 to the snare loop 114. The link groups 24 and 31 are disposed in the flexible sheath 113. A distal end of the elongated tube 108 is bent by pulling the control wire 143 to steer the snare loop 114 in one direction. When the handle section 131 is rotated, the link groups 24 and 31 transmit the rotation in a distal direction. The steering of the distal end can be changed in a direction as desired.

In the above embodiments, the torque transmission device 10 is an instrument for medical use. However, the torque transmission device 10 may be an instrument for any of the industrial fields in relation to a steerable device, steerable catheter, link assembly, robot arm, and the like.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A torque transmission device extending in a longitudinal axis direction, comprising:
a first link element having a first guide lumen;
a second link element, disposed at a distal end of said first link element in said longitudinal axis direction, and having a second guide lumen;
a control wire disposed through said first and second guide lumens movably in said longitudinal axis direction;
a first pivot mechanism for coupling said first link element to said second link element in a rotatable manner about a first pivot axis being perpendicular to said longitudinal axis direction;
wherein said control wire through said first and second guide lumens extends in a coplanar manner with said first pivot axis and crosswise thereto.

2. A torque transmission device as defined in claim 1, wherein said first pivot mechanism includes:
a first front connecting portion positioned on said first link element; and
a first rear connecting portion, positioned on said second link element, and engaged with said first front connecting portion in a rotatable manner.

3. A torque transmission device as defined in claim 1, further comprising:
a third link element, disposed at a distal end of said second link element in said longitudinal axis direction, having a third guide lumen, for receiving insertion of said control wire movably in said longitudinal axis direction;
a fourth link element, disposed at a distal end of said third link element in said longitudinal axis direction, having a fourth guide lumen, for receiving insertion of said control wire movably in said longitudinal axis direction;
a second pivot mechanism for coupling said third link element to said fourth link element in a rotatable manner about a second pivot axis being perpendicular to said longitudinal axis direction;
wherein said control wire through said third and fourth guide lumens extends in a non-coplanar manner with said second pivot axis and crosswise thereto.

4. A torque transmission device as defined in claim 3, wherein said second pivot mechanism includes:
a second front connecting portion positioned on said third link element; and
a second rear connecting portion, positioned on said fourth link element, and engaged with said second front connecting portion in a rotatable manner.

5. A torque transmission device as defined in claim 3, further comprising:
a distal link element, positioned in a distal end of a link group including said first to fourth link elements, and provided with a distal end of said control wire retained thereon;
a handle section, secured to a proximal end of said link group, for applying torque to said link group when rotated about an axis extending in said longitudinal axis direction; and
a wire puller, disposed on said handle section, secured to a proximal end of said control wire, for pulling said control wire when operated externally.

6. A torque transmission device as defined in claim 3, further comprising:
a fifth link element, disposed at a proximal end of said first link element in said longitudinal axis direction, having a guide lumen, for receiving insertion of said control wire movably in said longitudinal axis direction;
an additional pivot mechanism for coupling said fifth link element to said first link element in a rotatable manner about a predetermined pivot axis being perpendicular to said longitudinal axis direction;
wherein said predetermined pivot axis extends in a non-coplanar manner with said first pivot axis and crosswise thereto.

7. A torque transmission device as defined in claim 3, comprising:
a flexible section having said first and second link elements; and
a steering unit having said third and fourth link elements.

8. A torque transmission device as defined in claim 3, further comprising:
an intermediate link element for connecting said second link element with said third link element;
a third pivot mechanism for coupling said intermediate link element to said second link element in a rotatable manner about a third pivot axis extending crosswise to said control wire in a coplanar manner;
a fourth pivot mechanism for coupling said intermediate link element to said third link element in a rotatable manner about a fourth pivot axis extending in a non-coplanar manner with said control wire and crosswise thereto.

9. A torque transmission device as defined in claim 1, wherein said first pivot mechanism further includes a contact surface, formed on said first or second link element in a retracted manner, for allowing said first and second link elements to rotate relative to one another, said contact surface having a predetermined width in a first direction perpendicular to said first pivot axis, said predetermined width being equal to or more than a width of said first and second guide lumens in said first direction.

10. A torque transmission device as defined in claim 9, further comprising a coating, applied to said contact surface, for increasing resistance of friction.

11. A torque transmission device as defined in claim 9, further comprising a fine surface pattern of projections or recesses, formed on said contact surface, for increasing resistance of friction.

12. A torque transmission device as defined in claim 9, wherein said contact surface includes:
an arcuate surface; and
a flat surface formed by partially chamfering said arcuate surface.

13. A torque transmission device as defined in claim 5, further comprising a coating, applied to said contact surface, for increasing slipping property.

14. A torque transmission device as defined in claim 5, wherein said first pivot mechanism includes:
a connecting projection being arcuate when viewed in a section;
a connecting recess, being arcuate when viewed in a section, for receiving said connecting projection;
an inclined surface, formed with each of two walls of said connecting recess, for guiding said connecting projection toward said connecting recess.

15. A torque transmission device as defined in claim 5, wherein said first pivot mechanism includes:
a connecting projection being arcuate when viewed in a section;
a connecting recess, being arcuate when viewed in a section, for receiving said connecting projection;
a snap-fit structure for allowing push of said connecting projection into said connecting recess by resilient deformation for assembly, and for keeping said connecting projection rotatable in said connecting recess after said push.

16. A torque transmission device as defined in claim 1, wherein said torque transmission device is used with an endoscope and entered in a body cavity together with an elongated tube of said endoscope.

17. A torque transmission device as defined in claim 1, wherein said torque transmission device is incorporated in a high frequency snare instrument for medical use.
